(19) United States
(12) Plant Patent Application Publication
(10) Pub. No.: US 2007/0192914 P1
(43) Pub. Date: Aug. 16, 2007

(54) Orange tree named 'Alvarina'

(76) Inventor: Alvaro Sanchis Sisternes, Valencia (ES)

Correspondence Address: BUCHANAN, INGERSOLL & ROONEY PC, POST OFFICE BOX 1404, ALEXANDRIA, VA, 22313-1404, US

(21) Appl. No.: 11/705,713

(22) Filed: Feb. 14, 2007

(30) Foreign Application Priority Data
Feb. 16, 2006 (ES) 20064641

(51) Int. Cl. A01H 5/00

(52) U.S. Cl. PLT/202

(57) ABSTRACT

A new and distinct Citrus cultivar is provided which is capable of forming attractive large pleasantly tasting fruit having a light yellow-orange external coloration. The fruit when compared to that of the ‘Valencia Late’ Sweet Orange cultivar (non-patented in the United States) is lighter in coloration, generally more flattened, commonly possesses a greater diameter, and peels more readily. Also, the leaves tend to be larger, generally more rounded, and less elongated than those of the ‘Valencia Late’ cultivar. The fruit matures for consumption at approximately the same time as that of the ‘Valencia Late’ cultivar. The new cultivar is well suited for the commercial production of a late-maturing fruit crop.

### BOTANICAL/COMMERCIAL CLASSIFICATION

Citrus sinensis/Orange Tree

### VARIETAL DENOMINATION

cv. ALVARIÑA

### BACKGROUND OF THE NEW VARIETY

Citrus crops including Citrus sinensis Sweet Oranges are important to the agricultural economy in many parts of the world. Among Citrus plants commonly grown in California and Texas, U.S.A., is the ‘Valencia Late’ orange cultivar (non-patented in the United States) which is well recognized for its quality fruit.

The new cultivar of Citrus sinensis of the present invention was discovered during 1999 as a single branch mutation on a tree of the ‘Valencia Late’ cultivar at Valencia, Spain. The new cultivar is believed to be a significant mutation of the ‘Valencia Late’ cultivar of unknown causation.

I was primarily attracted to a single plant of the new cultivar in view of its distinctive fruit and leaf characteristics which were substantially different than those of the ‘Valencia Late’ cultivar. Had I not discovered and preserved the single branch of the new cultivar it would have been lost to mankind.

The new cultivar displays characteristics of both an Orange tree and a Tangarine tree.

It was found that the new Orange Tree of the present invention possesses the following combination of characteristics:
(a) forms attractive large seedless fruit which when compared to that of the ‘Valencia Late’ cultivar bears a lighter yellow-orange external coloration, a generally more flattened configuration, commonly a greater diameter, and peels more readily,
(b) forms larger leaves which generally are more rounded in configuration and less elongated than those of the ‘Valencia Late’ cultivar,
(c) bears late-maturing fruit that matures for consumption at approximately the same time as that of the ‘Valencia Late’ cultivar, and
(d) is well suited for the commercial production of a fruit crop.

The new cultivar of the present invention also can be readily distinguished from the ‘Nova’ cultivar (non-patented in the United States). More specifically, the ‘Nova’ cultivar forms smaller fruit, and the leaves are smaller and more elongated.

The new cultivar of the present invention has been found to undergo asexual propagation at Valencia, Spain, by the rooting of cuttings and by grafting. Such asexual propagation has been shown that the characteristics of the new cultivar are strictly transmissible from one generation to another. Accordingly, the new cultivar undergoes asexual propagation in a true to type manner.

The new cultivar has been named ‘Alvariña’.

### BRIEF DESCRIPTION OF THE PHOTOGRAPHS

The accompanying photographs show as nearly true as it is reasonably possible to make in color illustrations of this character typical specimens of the new variety. The plants were being grown at Valencia, Spain.

FIG. 1 illustrates a cluster of typical fruit wherein the light yellow-orange external coloration and the generally flattened configuration are illustrated.

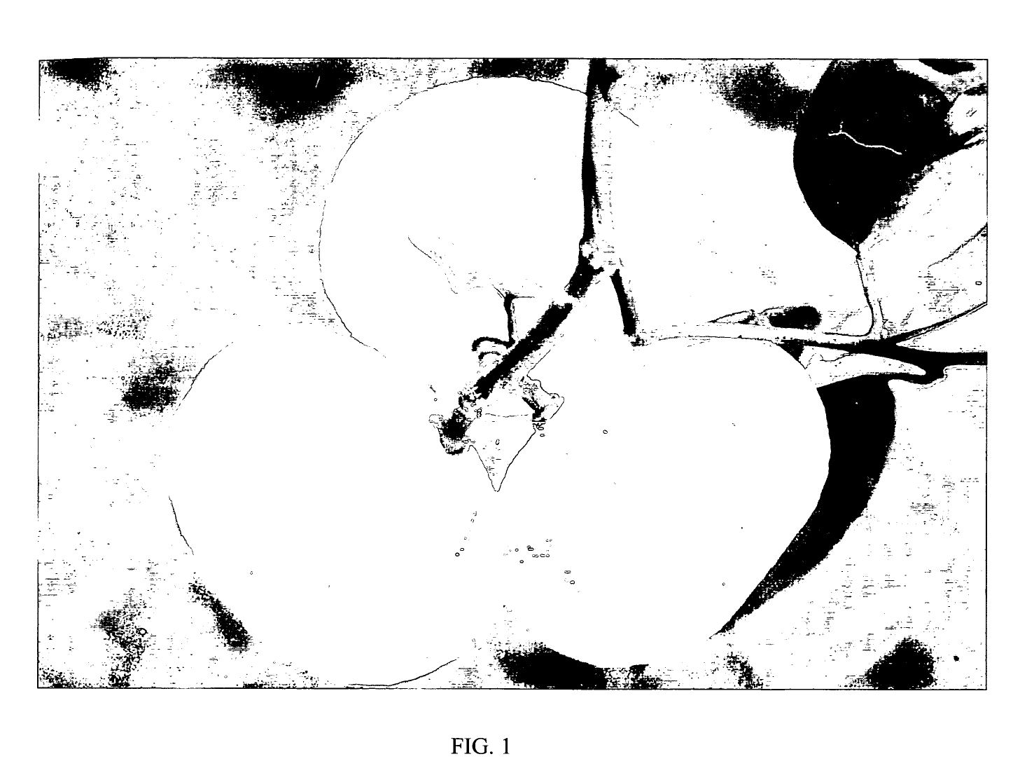

FIG. 2 illustrates the typical foliage of the new cultivar. The generally rounded configuration of the leaves is illustrated.

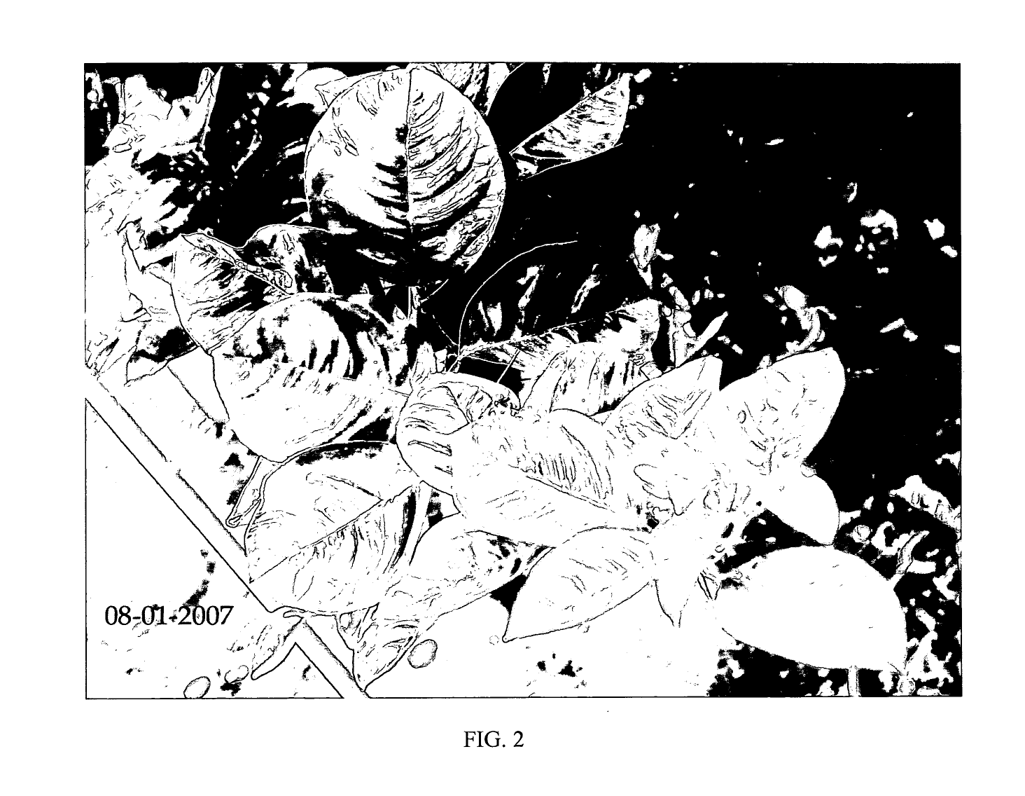

FIG. 3 illustrates the compact growth habit of a typical tree of the new cultivar while grafted on the ‘Navel Late’ cultivar (non-patented in the United States). The photograph was obtained on Jul. 15, 2005.

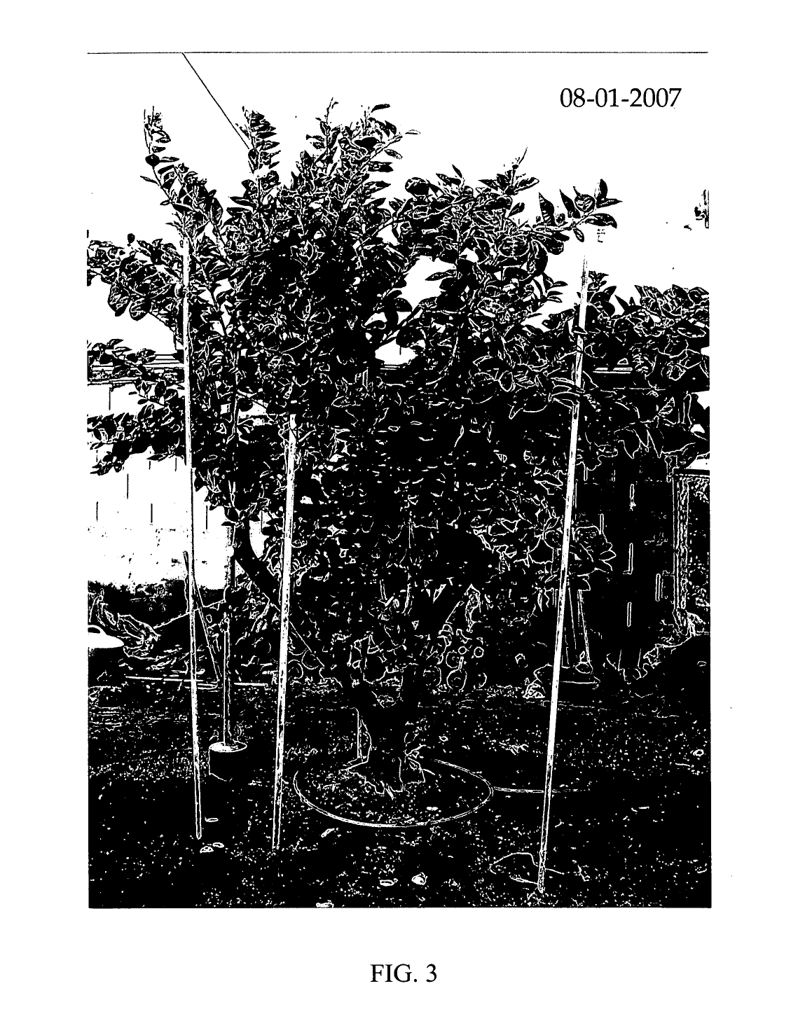

### DETAILED DESCRIPTION

The description is based on the observation of plants of the new cultivar growing outdoors at Valencia, Spain. Reference to color is to be accorded ordinary dictionary significance.
Classification: Citrus sinensis.
Tree:
   Growth habit.—vigorous and generally compact.
Leaves:
   Size.—large.
   Configuration.—large, rounded and generally elliptic, and larger and more rounded and less elongated than the leaves of the ‘Valencia Late’ cultivar.
   Apex.—generally cuspidate.
   Base.—cuneate to obtuse.
   Texture.—rugose with some glossiness.
   Coloration.—light to medium green.
Fruit:
   Time of maturity.—late, and matures for consumption at approximately the same time as the ‘Valencia Late’ cultivar.
   Size.—large.
   Configuration.—generally flattened unlike the ‘Valencia Late’ cultivar, commonly of a greater diameter than the ‘Valencia Late’ cultivar, and similar to that of a Tangerine.
   External coloration.—yellow-orange and generally lighter in coloration than the ‘Valencia Late’ cultivar.
   Internal flesh coloration.—light orange and generally comparable to that of the ‘Valencia Late’ cultivar.
   Ability to peel.—peels with ease when compared to the ‘Valencia Late’ cultivar.
   Naval.—absent or very rare.
   Parthenocarpy.—seedless fruit is formed.
   Eating quality.—excellent.

The new ‘Alvariña’ cultivar has not been observed under all possible environmental conditions to date. Accordingly, it is possible that the phenotypic expression may vary somewhat with changes in light intensity and duration, cultural practices, and other environmental conditions.

I claim:

1. A new distinct Citrus plant characterized by the following combination of characteristics:
(a) forms attractive large seedless fruit which when compared to that of the ‘Valencia Late’ cultivar bears a lighter yellow-orange external coloration, a generally more flattened configuration, commonly a greater diameter, and peels more readily,
(b) forms larger leaves which generally are more rounded in configuration and less elongated than those of the ‘Valencia Late’ cultivar,
(c) bears late-maturing fruit that matures for consumption at approximately the same time as the ‘Valencia Late’ cultivar, and
(d) is well suited for the commercial production of a fruit crop; substantially as herein shown and described.
